(19) United States
(12) Patent Application Publication
(10) Pub. No.: US 2008/0214517 A1
(43) Pub. Date: Sep. 4, 2008

(54) Modulation of the Expression of Estrogen Receptors for the Prevention or Treatment of Heart Disease

(76) Inventors: Jean-Francois Tanguay, Montreal (CA); Martin Sirois, Anjou (CA)

Correspondence Address: QUARLES & BRADY LLP, 411 E. WISCONSIN AVENUE, SUITE 2040, MILWAUKEE, WI, 53202-4497, US

(21) Appl. No.: 11/970,991

(22) Filed: Jan. 8, 2008

Related U.S. Application Data
(63) Continuation of application No. 10/910,130, filed on Aug. 2, 2004, now abandoned.
(60) Provisional application No. 60/491,257, filed on Jul. 31, 2003.

(51) Int. Cl. A61K 31/565; A61P 9/00; A61P 9/10

(52) U.S. Cl. 514/182

(57) ABSTRACT

The present invention relates to the upregulation of estrogen receptors (ER) alpha (ERα) and/or beta (ERβ) in endothelial cells and/or smooth muscle cells to prevent or treat heart disease. The upregulation is achieved through the use of recombinant DNA technology and, depending on therapeutic needs, may be performed with a simultaneous or subsquent downregulation, as with antisense technology. Oligonucleotides coding for ERα and/or ERβ are introduced into the targeted cells through the use of adenoviruses, for example. With an increase in receptors, the cells should be more responsive to such agonists as 17-beta-estradiol (17βE) and related compounds (genistein, estradiol derivatives . . . ) to improve plaque stabilization, vascular healing and endothelial recovery after vascular injury. Such oligonucleotides may be used to modulate the beneficial effects mediated by the ER on vascular healing, for example, restenosis or plaque stabilisation, in mammals. They may further be used in the prevention or treatment of a disease or disorder characterised by atherosclerosis, plaque vulnerability or destabilisation or pathological plaque rupture or erosion including spontaneous or induced injury.

### FIELD OF THE INVENTION

The present invention pertains to the modulation of the expression of mammalian estrogen receptors (ER) in order to enhance their effectiveness as cardioprotective, vascular healing and/or anti-atherosclerotic agents. This modulation may involve the upregulation of select ER or a combination of upregulation of a select ER and concurrent or subsequent downregulation of a different ER, according to the desired application.

### BACKGROUND OF THE INVENTION

Atherosclerosis is a process by which new lesions can progress or become vulnerable from pre-existing ones, and can be summarised as the culmination of i) increased endothelial cell dysfonction; ii) migration of inflammatory cells into extracellular matrix; iii) synthesis and release of degrading matrix molecules; iv) fibrous cap thinning, erosion and/or rupture; v) release of growth factors; vi) prothrombotic, proinflammatory, proapoptotic and/or proatherosclerotic status. Physiological vascular healing and regeneration are highly co-ordinated processes that occur in individuals under specific conditions, such as during spontaneous plaque rupture and/or destabilisation, or induced by percutaneous or surgical revascularization.

Estrogens play an important role in bone maintenance, in the cardiovascular system, in the growth, differentiation and biological activity of various tissues¹. The protective effects of 17-beta-estradiol (17βE) are related to favourable changes in plasma lipid profile, to inhibition of vascular smooth muscle cell (VSMC) proliferation³ and migration⁴, to relaxation of coronary vessels through endothelial nitric oxide synthase (eNOS) activity⁵, to reduction of platelets and monocyte aggregation⁶, tumor necrosis factor alpha (TNF-a) release⁷ and extracellular matrix synthesis⁸. It has been shown that local delivery of 17βE reduces neointimal thickness after coronary balloon injury in a porcine model⁸.

Estrogen can bind to two estrogen receptors (ER), alpha (ERα) and beta (ERβ), which are expressed in all vascular cells types⁹. The classical genomic mechanism, or long-term effect of estrogen on vascular tissues, is dependent on change in gene expression in the vascular tissues. Most recently, a second mechanism with direct (or nongenomic) estrogen effect has been identified¹⁰. Administration of estrogen can induce a rapid effect suggesting that its activities are linked to the induction of other intracellular pathways such as the mitogen-activated protein kinases (MAPKs)¹⁰. The MAPKs, which are involved in the proliferation, migration and differentiation of VSMC, are stimulated in rat carotid arteries after endothelial injury¹¹. Treatment with estrogen may influence the MAPK pathway in a variety of cell types and may provide protection against vascular injury.

As indicated above, the major effects of estrogens are mediated through two distinct estrogen receptors, ERα and ERβ. Each of these ER is encoded by a unique gene¹³ with some degree of homology between each other, and the genes are organized into six domains (A to F)⁹. The amino-terminal A-B domain represents the ligand-independent transcriptional-activation function 1 (TAF-1). The ER have only 18% of homology in this amino-terminus domain. The C domain, which represents the DNA binding domain, is extremely conserved in all steroid receptors and domain D contains the hinge region of the ER. The hormones bind the E domain which also contains a ligand-dependent transcriptional-activation function 2 (TAF-2). The two ER have 97% and 60% homology in domains C and E, respectively. The carboxy-terminal F domain is a variable region and it has been proposed that the F domain may play an important role in the different responses of ER to 17βE or selective ER modulators¹⁴. The expression pattern of the two ER are very different in many tissues and may suggest distinct responses in the presence of 17βE. Three studies with transgenic knock-out (KO) mice were done and the treatment with 17βE, in the absence of one of two ER (αERKO and βERKO) or both ER (αβERKO) prevented the formation of hyperplasia following carotid injury.

### SUMMARY OF THE INVENTION

International Patent Application No. PCT/CA02/02000 entitled “An Antisense Strategy to Modulate Estrogen Receptor Response (ER. Alpha and/or ER. Beta)”, which is hereby incorporated by reference, describes the specific effects of each ER on the different vascular cell types, which are the endothelial cells and the smooth muscle cells. This application teaches the beneficial effects of 17βE in vascular healing and endothelial recovery after vascular injury by selectively inhibiting the expression of one or both receptors.

In contrast to the above application, the present invention involves the upregulation of ER so as to enhance the beneficial effects of 17βE and other ER agonists on endothelial and smooth muscle cell proliferation and migration, possibly in combination with conventional chemotherapy. However, in certain therapeutic applications, it may be advantageous to combine selective upregulation of a given ER with a selective downregulation (through antisense technology, for example, as described in International Patent Application No. PCT/CA02/0200) of a different ER. For example, depending on the cardiac disorder, an upregulation of ERα in endothelial cells may be indicated along with a concurrent or subsequent downregulation of ERβ in smooth muscle cells. Different combinations of upregulation and downregulation of ER are possible and are included within the scope of the present invention.

The upregulation of the expression of estrogen receptors in endothelium and smooth muscle cells should result in an enhancement of the beneficial response of these cells to 17βE and other agonists. One mechanism by which this upregulation may be achieved is through transfection of the vascular cells with an adenoviral expression vector comprising a transgene expressing the protein of interest (here, estrogen receptors ERα and/or ERβ) or expressing a suitable transcription factor upregulating the expression of the endogenous protein of interest. This may prove to be an advantageous alternative over the simple use of a ligand to these receptors in modulating their responses.

An object of the present invention is therefore to provide a method of selectively increasing the quantity of ER in vascular cell types in order to increase the effect of therapeutic agents, such as 17βE. This may be useful in medical treatments for various diseases and disorders including, but not limited to, atherosclerosis, inflammation, plaque destabilisation, vascular injury and restenosis.

In accordance with one aspect of the invention, there is provided a method of modulating vascular healing after spontaneous, catheter or surgically induced injury in a mammal in need of such therapy, comprising the step of upregulating the expression of a gene encoding a mammalian ER selected from the group consisting of ERα and ERβ.

In accordance with another aspect of the invention, there is provided a method of preventing atherosclerotic plaque vulnerability or destabilisation in a mammal in need of such therapy, comprising the step of upregulating the expression of a gene encoding a mammalian ER selected from the group consisting of ERα and ERβ.

In accordance with a further aspect of the invention, there is provided a method of treating atherosclerotic plaque vulnerability or destabilisation in a mammal in need of such therapy, comprising the step of upregulating the expression of a gene encoding a mammalian ER selected from the group consisting of ERα and ERβ.

In accordance with yet another aspect of the invention, there is provided a method of reducing pathological angiogenesis in a mammal in need of such therapy, comprising the step of upregulating the expression of a gene encoding a mammalian ER selected from the group consisting of ERα and ERβ.

In accordance with a further aspect of the invention, there is provided a method of promoting saphenous vein graft healing in a mammal in need of such therapy, comprising the step of upregulating the expression of a gene encoding a mammalian ER selected from the group consisting of ERα and ERβ.

In accordance with another aspect of the invention, there is provided a method of blocking pathological vascular injury or vulnerability in a mammal in need of such therapy, comprising the step of upregulating the expression of a gene encoding a mammalian ER selected from the group consisting of ERα and ERβ. This upregulation is achieved by any suitable method involving recombinant DNA.

In accordance with yet another aspect of the invention, there is provided a method of improving vascular healing in a mammal in need of such therapy, comprising the step of upregulating the expression of a gene encoding a mammalian ER selected from the group consisting of ERα and ERβ. This upregulation is achieved by any suitable method involving recombinant DNA.

In a specific embodiment of the present invention, the expression of ERα receptors is increased in endothelial cells. In another specific embodiment of the present invention, the expression of ERβ receptors is increased in smooth muscle cells. In yet another specific embodiment of the present invention, the expression of ERα receptors is increased in endothelial cells and the expression of ERβ receptors is downregulated in smooth muscle cells.

Other objects, advantages and features of the present invention will become more apparent upon reading of the following non-restrictive description of preferred embodiments thereof, given by way of example only.

### BRIEF DESCRIPTION OF THE DRAWINGS

FIG. 1: Antisense regulation of ERα and ERβ expression on PSMC and PAEC. PSMC and PAEC were seeded at 1×10⁶ cells/100-mm culture plate and grown to confluence. Cells were treated either with antisense or scrambled oligomers as described in the methods. ERα (66 kDa) and ERβ (54 kDa) protein expression were detected by Western blot analyses. Image densitometry results are given as relative expression (%) as compared to control PBS-treated cells.

FIG. 2: Contribution of ERα and ERβ on PSMC proliferation. PSMC were seeded at 1×10⁴ cells/well and stimulated with or without antisense oligomers as described in the methods. Cells were then stimulated with or without 17βE (10⁻⁸ mol/L) and a cell count achieved 72 hours post-treatment. The values are means of cell counts obtained from 6 wells for each treatment. *, P<0.05 as compared to day 0; t, P<0.05 as compared to control (1% FBS); *, P<0.05 as compared to cells treated with 17βE (10% mol/L).

FIG. 3: Contribution of ERα and ERβ on PSMC migration. PSMC were trypsinized and resuspended in DMEM; 2.5×10⁵ cells/well of a six-well tissue culture plate were stimulated with or without antisense oligomers as described in the methods. 2.5×10⁴ cells were added in the higher compartment of the modified Boyden chamber apparatus with or without antisense oligomers, and the lower chamber was filled with DMEM 1% FBS, and antibiotics with or without PDGF-BB (10 ng/ml). Five hours postincubation at 37° C., the migrated cells were fixed, stained and counted by using a microscope adapted to a digitized video camera. The values are represented as relative mean of migrating cells from 6 chambers for each treatment. *, P<0.05 as compared unstimulated cells; †, P<0.05 as compared to cells treated with PDGF-BB; ‡, P<0.05 as compared to cells treated with 17βE (10⁻⁸ mol/L).

FIG. 4: Contribution of ERα and ERβ on p42/44 and p38 MAPK activation in PSMC. PSMC were seeded at 1×10⁶ cells/100-mm culture plate and grown to confluence. Cells were treated either with antisense or scrambled oligomers as described in the methods. Cells were then treated with or without 17βE (10⁻⁸ mol/L) for 30 minutes and stimulated 5 minutes for p42/44 MAPK (A) or 30 minutes for p38 MAPK (B) with PDGF-BB. Proteins were detected by Western blot analyses. Image densitometry results are given as relative expression (%) as compared to control PBS-treated cells.

FIG. 5: Contribution of ERα and ERβ on PAEC proliferation. PAEC were seeded at 1×10⁴ cells/well and stimulated with or without antisense oligomers as described in the methods. Cells were then stimulated with or without 17βE (10⁻⁸ mol/L) and a cell count achieved 72 hours post-treatment. The values are means of cell counts obtained from 6 wells for each treatment. *, P<0.05 as compared to day 0; †, P<0.05 as compared to control (1% FBS); ‡, P<0.05 as compared to cells treated with 17βE (10⁻⁸ mol/L).

FIG. 6: Contribution of ERα and ERβ on PAEC migration. PAEC were trypsinized and resuspended in DMEM; 2.5×10⁵ cells/well of a six-well tissue culture plate were stimulated with or without antisense oligomers as described in the methods. 2.5×10⁴ cells were added in the higher compartment of the modified Boyden chamber apparatus with or without antisense oligomers, and the lower chamber was filled with DMEM 1% FBS, and antibiotics with or without 17βE (10⁻⁸ mol/L). Five hours postincubation at 37° C., the migrated cells were fixed, stained and counted by using a microscope adapted to a digitized video camera. The values are represented as relative mean of migrating cells/mm² from 6 chambers for each treatment. *, P<0.05 as compared to unstimulated cells; t, P<0.05 as compared to cells treated with 17βE (10⁻⁸ mol/L).

FIG. 7: Contribution of ERα and ERβ on p42144 and p38 MAPK activation in PAEC. PAEC were seeded at 1×10⁶ cells/100-mm culture plate and grown to confluence. Cells were treated either with antisense or scrambled oligomers as described in the methods. Cells were then treated with or without 17βE (10⁻⁸ mol/L) for 5 minutes for p42/44 MAPK activation (A) or 30 minutes for p38 MAPK stimulation (B). Proteins were detected by Western blot analyses. Image densitometry results are given as relative expression (%) as compared to control PBS-treated cells.

### DETAILED DESCRIPTION OF THE INVENTION

The present invention employs recombinant technology for modulating the function of nucleic acid molecules encoding estrogen receptors ERα and ERβ, ultimately modulating the amount of ER protein produced. This is accomplished through the use of recombinant DNA technology that is known in the art, such as through the use of adenoviruses to transfect the target vascular cells. The overall effect of such interference with target nucleic acid function is modulation of the expression of estrogen receptors (ER), and specifically, a selective upregulation, of ERα and/or ERβ receptors.

The present invention therefore relates to a new prophylactic and therapeutic strategy for the prevention or treatment of heart disease. This strategy relies on the upregulation of ERα and/or ERβ. Depending on the nature of the intervention required, this upregulation may be accompanied by the administration of 17βE or a related compound (genistein, estradiol derivatives . . . ) in order to optimize the effects on vascular healing and endothelial recovery after vascular injury, for example. The recombinant DNA technology, alone or in combination with conventional chemotherapy, will improve vascular healing and endothelial recovery after vascular injury and will bring a new, innovative therapy with application not limited to cardiovascular angioplasty but in other areas of the body (cerebral, renal, peripheral vasculature . . . ) where estrogens have an effect.

### DEFINITIONS

Unless defined otherwise, all technical and scientific terms used herein have the same meaning as commonly understood by one of ordinary skill in the art to which this invention belongs, as exemplified by The McGraw-Hill Dictionary of Chemical Terms (Ed. Parker, S., 1985), McGraw-Hill, San Francisco).

“Naturally-occurring”, as used herein, as applied to an object, refers to the fact that an object can be found in nature. For example, a polypeptide or polynucleotide sequence that is present in an organism (including viruses) that can be isolated from a source in nature and which has not been intentionally modified in the laboratory is naturally-occurring.

“Nucleic acid” refers to DNA and RNA and can be either double stranded or single stranded. The invention also includes nucleic acid sequences which are complementary to the claimed nucleic acid sequences.

“Oligonucleotide”, as used herein, refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

“Polynucleotide” refers to a polymeric form of nucleotides of at least 10 bases in length, either ribonucleotides or deoxynucleotides or a modified form of either type of nucleotide. The term includes single and double stranded forms of DNA or RNA.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

“Protein”, as used herein, refers to a whole protein, or fragment thereof, such as a protein domain or a binding site for a second messenger, co-factor, ion, etc. It can be a peptide or an amino acid sequence that functions as a signal for another protein in the system, such as a proteolytic cleavage site.

“Transfection”, as used herein, refers to the introduction of DNA into a recipient eukaryote cell and its subsequent integration into the recipient cells chromosomal DNA. Usually accomplished using DNA precipitated with calcium ions though a variety of other methods can be used (e.g. electroporation, adenovirus systems, nanoparticles, liposomes and microspheres). Transfection is analogous to bacterial transformation but in eukaryotes transformation is used to describe the changes in cultured cells caused by tumour viruses, for example.

An expression vector comprising the sense oligonucleotide sequence may be constructed by using procedures known in the art.

Vectors can be constructed by those skilled in the art to contain all the expression elements required to achieve the desired transcription of the desired ER oligonucleotide sequences. Therefore, the invention provides vectors comprising a transcription control sequence operatively linked to a sequence which encodes an ER oligonucleotide to increase the synthesis of the ER so as to upregulate their production. Suitable transcription and translation elements may be derived from a variety of sources, including bacterial, fungal, viral, mammalian or insect genes. Selection of appropriate elements is dependent on the host cell chosen.

Within the context of the present invention, a possible intragenic site is the region encompassing the translation initiation or termination codon of the open reading frame (ORF) of the gene. It is known in the art that eukaryotic and prokaryotic genes may have two or more alternative start codons, any one of which may be utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding a mammalian estrogen receptor (ER) that is ERα or ERβ, regardless of the sequence(s) of such codons.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′cap of an mRNA comprises an N⁷-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also potential targets. It has also been found that introns can be effective target regions for antisense compounds targeted, for example, to DNA or pre-mRNA.

Alternative modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphos-phonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Alternative modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In alternative oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al (1991) Science, 254, 1497-1500.

Modified oligonucleotides may also contain one or more substituted sugar moieties. For example, oligonucleotides may comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘ CH₃, O(CH₂)ₙ OCH₃, O(CH₂)ₙ NH₂, O(CH₂)ₙ CH₃, O(CH₂)ₙONH₂, and O(CH₂)ₙ ON[(CH₂)ₙ CH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

Other modifications include 2′-methoxy (2′-O—CH₃), 2′-aminopropoxy (2′-OCH₂ CH₂ CH₂ NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al (1991) Angewandte Chemie, International Edition, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278), even more particularly when combined with 2′-O-methoxyethyl sugar modifications.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al (1989) Proc. Natl. Acad. Sci. USA, 86, 6553-6556), cholic acid (Manoharan et al (1994) Bioorg. Med. Chem. Lett., 4, 1053-1060), a thioether, e.g., hexyl-S-tritylthiol (Manoharan et al (1992) Ann. N.Y. Acad. Sci., 660, 306-309; Manoharan et al (1993) Bioorg. Med. Chem. Lett., 3, 2765-2770), a thiocholesterol (Oberhauser et al (1992) Nucl. Acids Res., 20, 533-538), an aliphatic chain, e.g., dodecandiol or undecyl residues (Saison-Behmoaras et al (1991) EMBO J., 10, 1111-1118), a phospholipid, e.g., di-hexadecyl-rac-glycerol or triethylammonium 1,2-di-O-hexadecyl-rac-glycero-3-H-phosphonate (Manoharan et al (1995) Tetrahedron Lett., 36, 3651-3654; Shea et al (1990) Nucl. Acids Res., 18, 3777-3783), a polyamine or a polyethylene glycol chain (Manoharan et al (1995) Nucleosides & Nucleotides, 14, 969-973), or adamantane acetic acid (Manoharan et al (1995) Tetrahedron Lett., 36, 3651-3654), a palmityl moiety (Mishra et al (1995) Biochim. Biophys. Acta, 1264, 229-237), or an octadecylamine or hexylamino-carbonyl-oxycholesterol moiety (Crooke et al (1996) J. Pharmacol. Exp. Ther., 277, 923-937.

It is not necessary for all positions in a given compound to be uniformly modified, and in fact more than one of the aforementioned modifications may be incorporated in a single compound or even at a single nucleoside within an oligonucleotide.

The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

The compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

Methods of delivery of foreign nucleic acids, such as oligonucleotides expressing ERα or ERβ, are known in the art, such as containing the nucleic acid in a liposome and infusing the preparation into an artery (LeClerc G. et al., (1992) J Clin Invest. 90: 936-44), transthoracic injection (Gal, D. et al., (1993) Lab Invest. 68: 18-25), and reliance on transfection techniques, such as the use of an adenovirus system. Other methods of delivery may include intravenous or intra-arterial administration of suitable ER DNA, or during catheterization procedures using any accepted device with the foreign ER DNA and inflating the balloon in the region of arteriosclerosis, thus combining balloon angioplasty and gene therapy (Nabel, E. G. et al., (1994) Hum Gene Ther. 5:1089-94). Such methods are known to those of skill in the art and can be tailored in accordance with the specific requirements of selected therapeutic interventions.

Another method of delivery involves “shotgun” delivery of the naked ER oligonucleotides across the dermal layer. The delivery of “naked” oligonucleotides is well known in the art. See, for example, Feigner et al., U.S. Pat. No. 5,580,859. It is contemplated that the ER oligonucleotides may be packaged in a lipid vesicle before “shotgun” delivery of the oligonucleotides.

Another method of delivery involves the use of electroporation to facilitate entry of the nucleic acid into the cells of the mammal. This method can be useful for introducing ER oligonucleotides to the cells to be treated, for example, endothelial cells, since the electroporation would be performed at selected treatment areas.

In one embodiment of the present invention the oligonucleotides or the pharmaceutical compositions comprising the oligonucleotides may be packaged into convenient kits providing the necessary materials packaged into suitable containers.

Cardiovascular diseases (CVD) are the leading cause of mortality for postmenopausal women in industrialized countries, accounting for more than 30% of deaths.¹⁵ Epidemiological studies over the past years suggested a protective effect of hormonal replacement therapy (HRT).¹⁶ Beneficial effects of estrogens were initially attributed to a decreased level of low-density lipoprotein cholesterol and to an increased level of high-density lipoprotein cholesterol. However, the positive effects of estrogens on lipid profile account about for only one-third of the observed reduction on the risk of mortality from CVD among HRT users.¹⁷ Other studies demonstrated that estrogens have direct actions on the blood vessel wall.¹⁸ Physiological concentrations of estrogens can inhibit platelet and monocyte aggregations, stimulate nitric oxide (NO) production and reendothelialization.¹⁹ Despite beneficial effects of estrogens, randomized double-blind studies reported no overall benefit from HRT.^{20.21} A better understanding of estrogen effects on vascular cells might contribute to optimize the vascular healing process.

Estrogen receptors (ERα and ERβ) are members of the steroid/thyroid hormone receptor superfamily of ligand-activated transcription factors.²² Estrogen receptors contain DNA and ligand binding domains which are critically involved in regulating vascular structures and functions.²³ Receptor-ligand interactions trigger a cascade of events including dissociation from heat shock proteins, receptor dimerization, phosphorylation and the association of the hormone activated receptor with specific regulatory elements in target genes.²³ ERα and ERβ are expressed in vascular endothelial (EC) and smooth muscle cells (SMC) and their activation may lead to distinct biological activities even though they share many functional characteristics.²⁴ In a previous study, Pare et al²⁵ showed in ERα and ERβ knockout mice that the protective effects of estrogens to vascular injury are ERα-dependent. However, the exact contribution played by ERβ remains to be clarified. Previous experiments showed that ERβ-deficient mice exhibit higher vasoconstriction and blood pressure as compared to wild-type mice.²⁶ However, several limitations exist when using knock-out animal preparation whereas a disruption of a gene may influence the response of estrogens.

Recently, we reported that a local delivery of 17-beta-estradiol (17βE) following a coronary angioplasty in pigs promoted the vascular healing process by reducing neointimal formation, and by improving the reendothelialization process, and the endothelial NO synthase (eNOS) expression.^{27.28} Classically, ER act as transcriptional factor by regulating the gene expression. However, other specific effects of estrogens may induce nongenomic signalling pathways and may interact with intracellular second messengers such as mitogen-activated protein kinase (MAPK).²⁹ Under in vitro conditions, we showed that 17βE prevents SMC proliferation and migration by inhibiting p42/44 and p38 MAPK activation whereas it promotes these events in EC.³⁰ However, the specific contribution of ERα and ERβ on these events remains unknown. We used an antisense gene therapy approach to regulate the protein expression of ERα and ERβ and to better understand their specific contribution of each ER. Herein, we report that 17βE promotes p42/44 and p38 MAPK phosphorylation through ERα stimulation on EC, whereas on SMC the inhibitory effects of 17βE on p42/44 and p38 MAPK phosphorylation are mediated by ERβ activation.

### Materials and Methods

### Cell Culture

Porcine aortic endothelial cells (PAEC) and porcine smooth muscle cells (PSMC) were isolated from freshly harvested aortas, cultured and characterized as described previously.³⁰ PAEC and PSMC were used between passages 3 and 8.

### Antisense Oligonucleotide Gene Therapy

To distinguish the role played by ERα and ERβ on the migration and proliferation of PSMC and PAEC as well as on the activation of p38 and p42/44 MAPKs, we treated the cells with antisense oligonucleotide sequences complementary to porcine ERα and ERβ mRNA (GeneBank accession numbers Z37167 and AF164957, respectively). A total of 4 different antisense oligodeoxyribonucleotide phosphorothioate sequences were used, 2 targeting the porcine ERα mRNA (antisense 1, AS1-ERα: 5′-CTC GTT GGC TTG GAT CTG-3′; antisense 2: AS2-ERα: 5′-GAC GCT TTG GTG TGT AGG-3′), and 2 targeting the porcine ERβ mRNA (antisense 1, AS1-ERβ: 5′-GTA GGA GAC AGG AGA GTT-3′; antisense 2: AS2-ERβ: 5′-GCT AAA GGA GAG AGG TGT-3′). Two scrambled oligodeoxyribonucleotide phosphorothioate sequences (scrambled ERα, SCR-ERα: 5′-TGT AGC TCG GTT CTG TCG-3′; scrambled ERβ, SCR-ERβ: 5′-GAG TGG ACG TGA AGA AGT-3′) were also used as negative controls. These sequences were selected as they had no more than 3 consecutive guanosines, and with no or minimal capacity to dimerize together and to form hairpins. All sequences were synthesized at the Armand Frappier Institute (Laval, QC, Canada). Upon synthesis, the oligonucleotides were dried, resuspended in sterile water, and quantified by spectrophotometry.

Western Blot Analyses of ERα and ERβ Expression, p42/44 and p38 MAPK Phosphorylation

The efficiency and specificity of our antisense oligomers to prevent the expression of targeted proteins were evaluated by Western blot analyses. Culture medium of confluent PAEC and PSMC (100-mm tissue culture plate) was removed, the cells were rinsed with Dulbecco's modified eagle medium (DMEM; Life Technologies Inc., Carlsbad, Calif.) and trypsinized (trypsine-EDTA; Life Technologies). Cells were resuspended in DMEM containing 5% of fetal bovine serum (FBS) (Hyclone Laboratories, Logan, Utah) and antibiotics (penicillin and streptomycin, Sigma, St-Louis, Mo.), and a cell count was obtained with a Coulter counter Z1 (Coulter Electronics, Luton, UK). Cells were seeded at 1×10⁶ cells/100-mm tissue culture plate (Becton-Dickinson, Rutherford, N.J.), stimulated for 24 hours in DMEM, 5% FBS, and antibiotics with or without antisense oligomers (10⁻⁷, 5×10⁻⁷, 10⁻⁶ mol/L). LipofectAmine (5 μg/mL, Life Technology Inc.) was used to improve the cellular uptake of antisense oligomers in PSMC. Go synchronization was achieved by starving the cells for 48 hours in DMEM, 0.1% FBS, and antibiotics with or without antisense oligomers (10⁻⁷, 5×10⁻⁷, 10⁻⁶ mol/L) added daily. The cells were then grown to confluence for 16 hours in DMEM, 5% FBS, and antibiotics and starved for 7 hours in DMEM, 0.1% FBS, and antibiotics with or without antisense oligomers (10⁻⁷, 5×10⁻⁷, 10⁻⁶ mol/L) to induce an upregulation of the estrogen receptor expression. Culture media was removed, and the cells were rinsed. PSMC and PAEC were then stimulated with or without 17βE as previously described.¹⁶ Briefly, PSMC were incubated on ice in DMEM with or without 17βE (10⁻⁸ mol/L) for 30 minutes and incubated at 37° C. for 30 minutes. Cells were then rinsed, incubated in DMEM with PDGF-BB (10 ng/mL) for 30 minutes on ice, incubated at 37° C. for 5 or 30 minutes. PAEC were incubated on ice in DMEM with or without 17βE (10⁻⁸ mol/L) for 30 minutes, then incubated at 37° C. for 5 or 30 minutes. Total proteins were isolated by the addition of 500 μL of lysis buffer containing leupeptin 10 μg/mL (Sigma), phenylmethylsulfonyl fluoride 1 mmol/L (Sigma), aprotinin 30 μg/mL (Sigma), and NaVO₃ 1 mmol/L (Sigma). Plates were incubated at 4° C. for 30 minutes and scraped, and the protein concentration was determined with a Bio-Rad protein assay kit (Bio-Rad, Hercules, Calif.). Proteins (100 μg) were separated by a 10% gradient SDS-polyacrylamide gel electrophoresis (SDS-PAGE) (Protean II kit; Bio-Rad), and transblotted onto a 0.45-μm polyvinylidene difluoride membranes (Millipore Corp., Bedford, Mass.). The membranes were blocked in 5% Blotto-TTBS (5% nonfat dry milk (Bio-Rad), 0.05% Tween 20, 0.15 mol/L NaCl, 25 mmol/L Tris-HCl, pH 7.4) for 1 hour at room temperature with gentle agitation and incubated overnight in 0.5% Blotto-TTBS containing the desired antibody (rabbit polyclonal anti-human-ERα or anti-human-ERβ; 1:5000 dilution, Santa Cruz Biotechnology, Santa Cruz, Calif.; or rabbit polyclonal anti-phospho-p42/44 MAPK; 1:10000 dilution, or anti-phospho-p38 MAPK; 1:5000 dilution, New England BioLabs, Beverly, Mass.). Membranes were washed 3 times with TTBS, and incubated with a horseradish peroxidase goat anti-rabbit IgG antibody (1:10000 dilution, Santa Cruz Biotechnology) in 0.5% Blotto-TTBS for 30 minutes. Membranes were washed with TTBS, and horseradish peroxidase bound to secondary antibody was revealed by chemiluminescence (Renaissance kit, NEN Life Science Products, Boston, Mass.). Kaleidoscope molecular weight and SDS-PAGE broad range marker proteins (Bio-Rad) were used as standards. Digital image densitometry (PDI Bioscience, Aurora, ON) was performed to determine the relative expression of ERα and ERβ proteins. Western blot analyses were performed in triplicate and results of image densitometry are representative of these experiments.

### Mitogenic Assay

Confluent PAEC and PSMC were rinsed with DMEM and trypsinized. Cells were resuspended in 10 mL of DMEM, 5% FBS, and antibiotics, and a cell count was obtained by using a Coulter counter Z1. PAEC and PSMC were initially seeded at 1×10⁴ cells/well of 24-well tissue culture plates stimulated for 24 hours in DMEM, 5% FBS, and antibiotics with or without antisense oligomers (10⁻⁶ mol/L), and starved for 48 hours in DMEM, 0.1% FBS, and antibiotics with or without antisense oligomers (10⁻⁶ mol/L daily) for Go synchronization. The cells were stimulated for 72 hours in DMEM, 1% FBS, antibiotics with or without antisense oligomers (10⁻⁶ mol/L daily) and with or without of 17βE (10⁻⁸ mol/L). After trypsinization, cell number was determined by using a Coulter counter Z1.

### Chemotactic Assay

Cell migration was evaluated using a modified Boyden 48-well microchamber kit (NeuroProbe, Cabin John, Md.). Near confluent PAEC and PSMC were rinsed with DMEM and trypsinized. Cells were resuspended in DMEM, 5% FBS, and antibiotics, and a cell count was obtained. PAEC and PSMC were seeded at 2.5×10⁵ cells/well of 6-well tissue culture plates; stimulated for 24 hours in DMEM, 5% FBS, and antibiotics with or without antisense oligomers (10⁶ mol/L) and starved for 48 hours in DMEM, 0.1% FBS, and antibiotics with or without antisense oligomers (1-6 mol/L daily) with or without 17βE (10⁻⁸ mol/L). Cells were harvested by trypsinization and resuspended in DMEM, 1% FBS, and antibiotics at a concentration of 2.5×10⁴ cells/mL. Fifty μL of this cell suspension with or without antisense oligomers (10⁻⁶ mol/L) treated with or without 17βE (10⁻⁸ mol/L) was added in the higher chamber of the modified Boyden chamber apparatus, and the lower chamber was filled with DMEM, 1% FBS, antibiotics plus the desired concentration of agonist either 17βE (10⁻⁸ mol/L) or platelet-derived growth factor-BB (PDGF-BB). The 2 sections of the system were separated by a porous polycarbonate filter (5-μm pores size), pretreated with a gelatine solution (1.5 mg/mL), and assembled. Five hours postincubation at 37° C., the nonmigrated cells were scraped with a plastic policeman, and the migrated cells were stained using a Quick-Diff solution (Shandon Inc, Pittsburgh, Pa.). The filter was then mounted on a glass slide, and migrated cells were counted using a microscope adapted to a video camera to obtain a computer-digitized image. Because of slight variation of basal cell migration between experiments, data were reported as relative mean migrating cells compared to baseline.

### Statistical Analysis

Data are mean=SEM. Statistical comparisons were performed using ANOVA followed by a multiple comparisons Bonferroni correction. A P value<0.05 was considered as significant.

### Results

### Modulation of ERα and ERβ Protein Expression by Antisense Oligonucleotide Gene Therapy

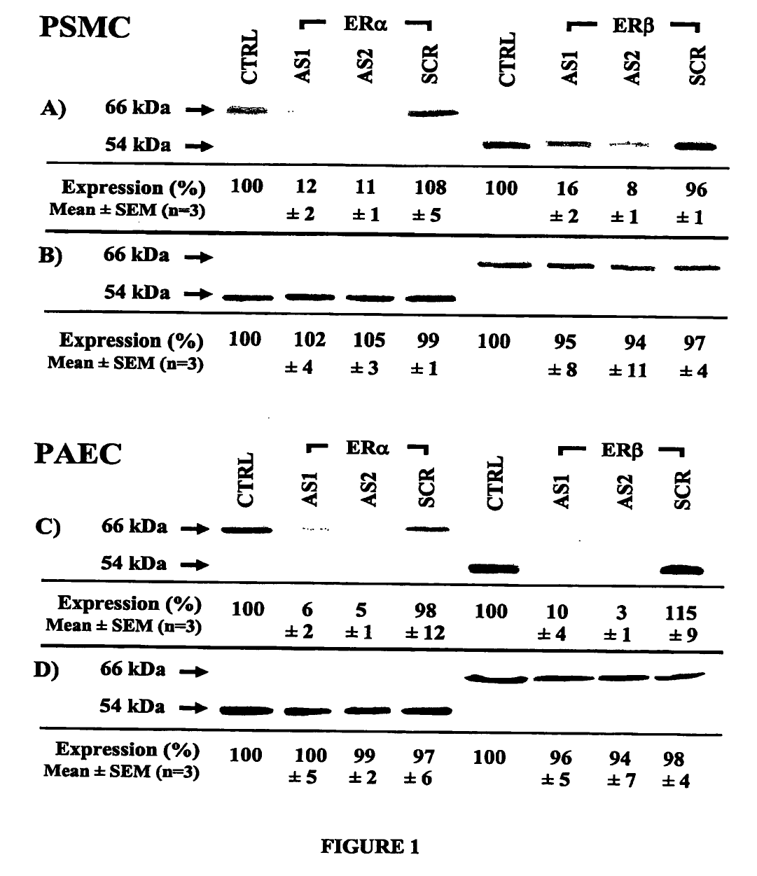

In order to evaluate the potency of antisense oligonucleotides to prevent the expression of targeted proteins, PSMC and PAEC were treated either with antisense or scrambled oligomers, and the expression of each receptor determined by Western blot analyses. In PSMC, we observed a basal ERα protein expression (Ctrl) which was inhibited by a treatment with antisense oligomers (10⁻⁶ mol/L) targeting porcine ERα mRNA. The antisense oligomers AS1-ERα and AS2-ERα suppressed ERα protein expression by 88 and 89% in PSMC, respectively (FIG. 1A). Similar treatment with antisense oligomers (AS1-ERβ and AS2-ERβ; 10⁻⁶ mol/L) directed against ERβ mRNA reduced also the basal ERβ protein expression in PSMC by 84 and 92%, respectively (FIG. 1A). The same series of experiments was conducted in PAEC. The antisense oligomers AS1-ERα and AS2-ERα (10⁻⁶ mol/L) suppressed PAEC ERα protein expression by 94 and 95%, respectively (FIG. 1B) and AS1-ERβ and AS2-ERβ (10⁻⁶ mol/L) downregulated ERβ protein expression by 90 and 97%, respectively (FIG. 1C). Treatment with scrambled oligomers (SCR-ERα and SCR-ERβ; 10⁻⁶ mol/L) had no significant effect on basal ERα and ERβ protein expression (FIGS. 1A and 1B).

To ensure that the antisense oligomers designed to downregulate the expression of ERα would not affect ERβ expression and vice versa, we performed additional Western blot analyses to evaluate the specificity of our antisense oligomers. Treatment with antisense oligomers targeting ERα mRNA (10⁻⁶ mol/L) did not affect ERβ basal protein expression while the antisense oligomers directed against ERβ, mRNA (10⁻⁶ mol/L) did not alter the basal protein expression of ERα on PSMC and PAEC (FIGS. 1B and 1D).

### Contribution of ERα and ERβ on PSMC Proliferation

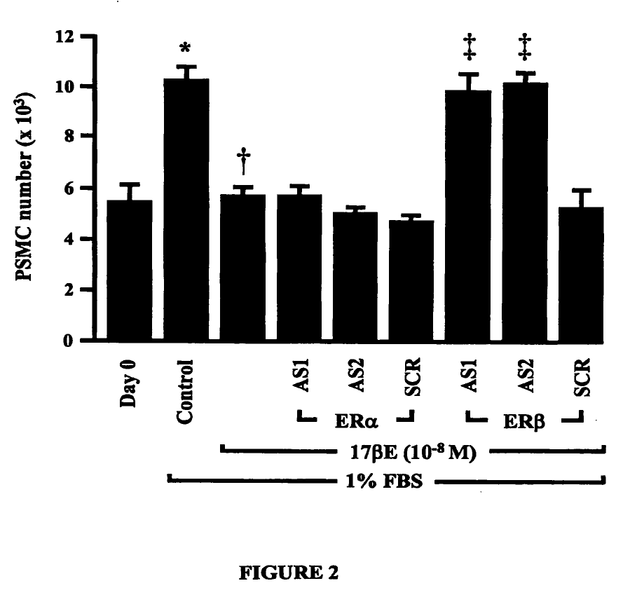

As the expressions of ERα and ERβ were specifically blocked by antisense oligomers, we investigated the contribution of both receptors on PSMC proliferation. Stimulation of quiescent PSMC with DMEM 1% FBS for 72 hours increased PSMC proliferation by 88% from 5432±680 cells/well to 10216±546 cells/well (FIG. 2). Treatment with 17βE (10 mol/L) prevented by 95% the PSMC proliferation mediated by FBS 1%. Treatment of PSMC with AS1-ERβ and AS2-ERβ prevented the inhibitory effects of 17βE on PSMC proliferation (P<0.05), while the antisense oligomers directed against ERα mRNA did not influence 17βE activity (FIG. 2). Treatment with scrambled oligomers did not affect the inhibitory activity of 17βE on PSMC proliferation (FIG. 2).

### Anti-Chemotactic Effect of 17βE on PSMC: Role of ERα and ERβ

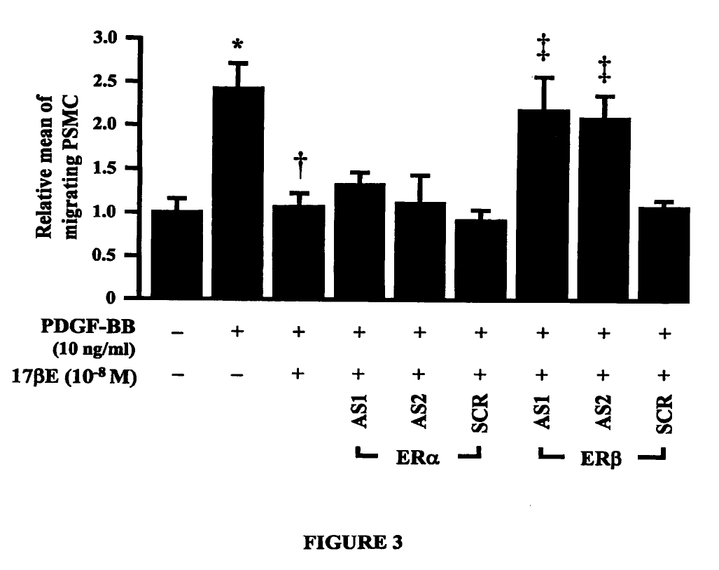

By using a modified Boyden chamber assay, we observed that a treatment with PDGF-BB (10 ng/mL) for 5 hours increased the basal migration of PSMC by 141% as compared to cells treated with FBS 1% (FIG. 3). Treatment with 17βE (10⁻⁸ mol/L) inhibited completely the chemotactic effect of PDGF-BB (10 ng/mL) (FIG. 3). In order to evaluate the contribution of each ER subtype on 17βE anti-chemotactic effect on PSMC, the cells were treated with antisense oligomers targeting either ERα or ERβ mRNA. Treatment with antisense against ERα mRNA (10⁻⁶ mol/L) did not alter the effect of 17βE on PSMC migration induced by PDGF-BB. However, a treatment with AS1-ERβ and AS2-ERβ directed against ERβ mRNA abolished the anti-chemotactic effect of 17βE on PSMC (P<0.05). Treatment with scrambled oligomers did not influence the 17βE anti-chemotactic activity on PSMC (FIG. 3).

Role of ERα and ERβ on p42/44 and p38 MAPK Phosphorylation in PSMCs

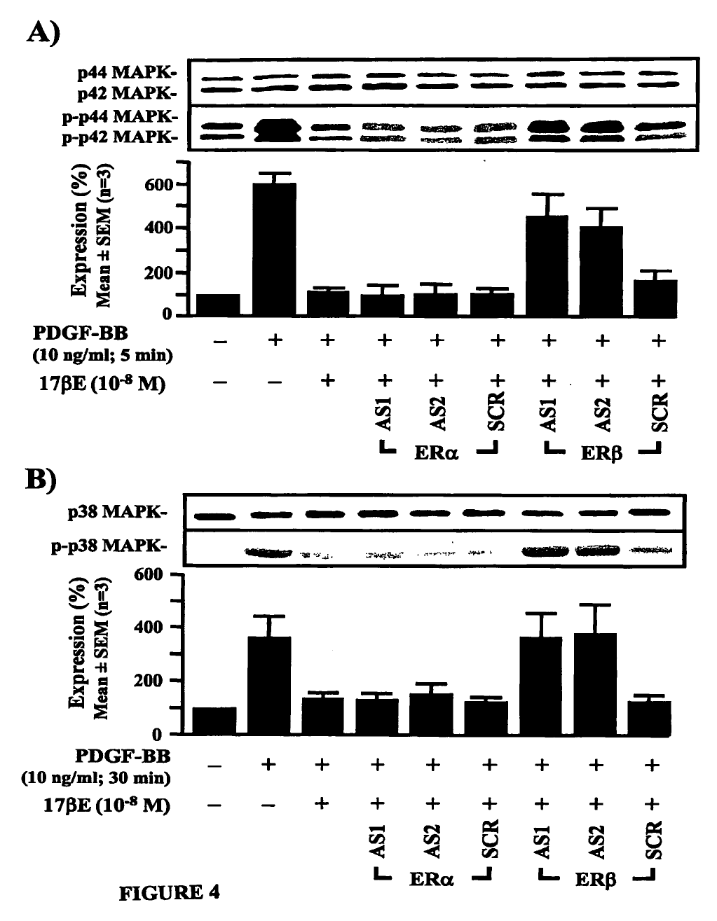

As 17βE can influence p42/44 and p38 MAPK phosphorylation in PSMC, we evaluated the specific contribution of ERα and ERβ in this regard. Treatment of PSMC with PDGF-BB increased p42/44 (FIG. 4A) and p38 MAPK phosphorylation (FIG. 4B) which was reversed by a 30-minute pretreatment with 17βE (10⁻⁸ mol/L). Treatment of PSMC with antisense oligomers targeting ERα mRNA did not affect the inhibitory effect of 17βE at preventing p42/44 and p38 MAPK phosphorylation induced by PDGF-BB. In contrast, a treatment with antisense oligomers directed against ERβ mRNA blocked significantly the effects of 17βE on p42/44 and p38 MAPK phosphorylation (P<0.05) (FIGS. 4A and 4B). In the same series of experiments, scrambled oligomers did not alter 17βE activity on these MAPKs (FIGS. 4A and 4B).

### Contribution of ERα and ERβ on PAEC Proliferation

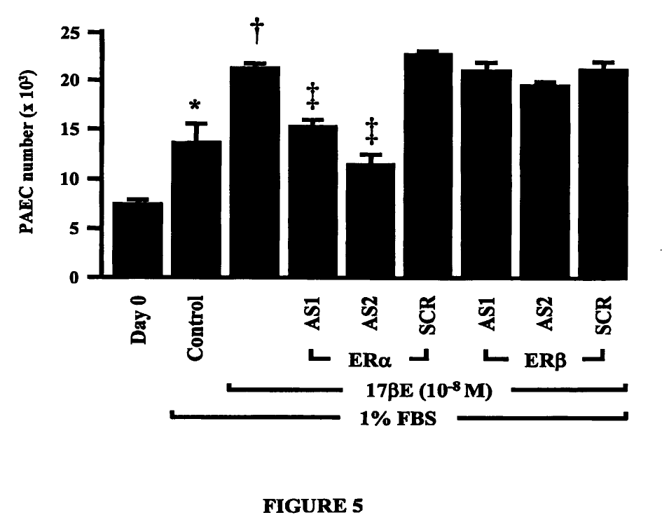

Stimulation of PAEC with DMEM 1% FBS increased their proliferation by 83% from 7427±423 to 13566±1931 cells/well within 3 days. The addition of 17βE (10⁻⁸ mol/L) enhanced the proliferation of PAEC by 123% as compared to the cells treated with FBS 1% (FIG. 5). To investigate the selective contribution of ERα and ERβ on the positive mitogenic effect of 17βE on endothelial cells, PAEC were treated with antisense oligomers targeting ERα or ERβ mRNA. AS1-ERα and AS2-ERα reduced significantly the mitogenic effects of 17βE by 80 and 100%, respectively (P<0.05). Treatment with antisense oligomers directed against ERβ mRNA failed to alter the mitogenic activity of 17βE on PAEC. Again, PAEC proliferation induced by 17βE was not influenced by treatments with scrambled antisense oligomers (FIG. 5).

Anti-Chemotactic Effects of 17βE on PAEC: Role of ERα and ERβ mRNA

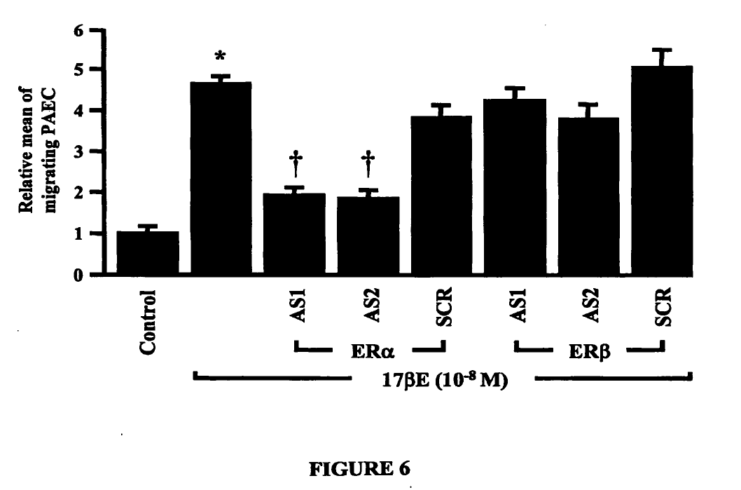

Treatment of PAEC with 17βE (10⁻⁸ mol/L) for 5 hours promoted their migration by 363% as compared to cells treated with FBS 1% (P<0.05) (FIG. 6). Treatment with antisense oligomers (10⁻⁶ mol/L) directed against ERα mRNA prevented the chemotactic activity of 17βE (10⁻⁸ mol/L) on PAEC by 75 and 76%, respectively (P<0.05) (FIG. 6), whereas the inhibition of ERβ protein expression did not prevent the 17βE activity on PAEC (FIG. 6). Treatment with scrambled oligomers did not alter the chemotactic activity of 17βE (FIG. 6).

Role of ERα and ERβ on p42/44 and p38 MAPK Phosphorylation In PAECs

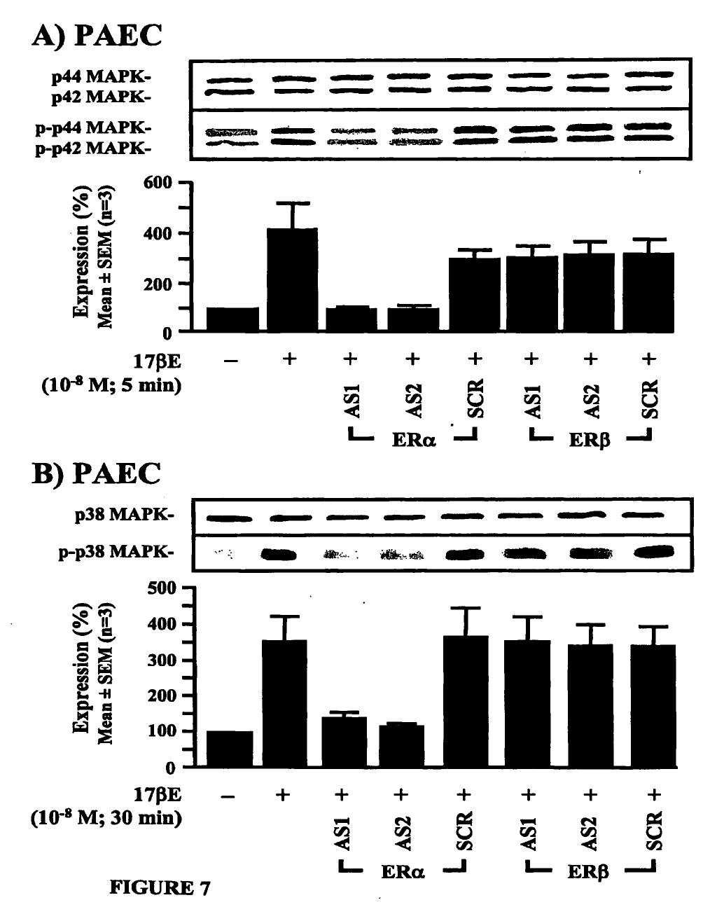

We have previously demonstrated that 17βE induces a marked increase of p42/44 and p38 MAPK phosphorylation in PAEC. In order to determine the contribution of ERα and ERβ on these intracellular mechanisms, PAEC were treated with antisense oligomers targeting ERα or ERβ mRNA. PBS-treated PAEC showed a basal phosphorylation of p42/44 (FIG. 7A) and p38 MAPK (FIG. 7B). Stimulation with 17βE (10⁻⁸ mol/L) for 5 minutes increased p42/44 MAPK phosphorylation by 317%, and 30 minutes stimulation with 17βE increased p38 MAPK phosphorylation by 254%. Treatment of PAEC with AS1-ERα and AS2-ERα prevented p42/44 and p38 MAPK phosphorylation induced by 17βE (FIGS. 7A and 7B). In contrast, treatment with antisense oligomers targeting ERβ mRNA did not reduce significantly p42/44 and p38 MAPK phosphorylation mediated by 17βE. Treatment with scrambled oligomers did not influence 17βE activity on p42/44 and p38 MAPK phosphorylation (FIGS. 7A and 7B).

Previous studies have demonstrated that the disruption of ERα in mice reduces the cardioprotective effects of estrogens on restenosis.²⁵ However, other investigators have indicated that ERβ, the major ER expressed within the vasculature, might contribute to the beneficial effects of estrogen Previously, we demonstrated that a local delivery of 17βE upon a porcine coronary angioplasty reduces restenosis by improving the reendothelialization process, the eNOS expression and the vascular healing.^{27,28} In addition, we showed under in vitro conditions that the beneficial effects of 17βE on restenosis may be explained by a reduction of PSMC p38 and p42/44 MAPK phosphorylation, migration and proliferation combined to a positive effect of these mechanisms in PAEC.³⁰ To the best of our knowledge, the specific contribution of each ER (ERα and ERβ) on MAPK phosphorylation and vascular cell migration and proliferation remained unknown. In the current study, we demonstrated that these effects of 17βE on PAEC are mediated through ERα activation whereas, in PSMC, 17βE activities are mediated through ERβ stimulation.

### Regulation of ERα and ERβ Protein Expression by Antisense Gene Therapy

We used an antisense gene therapy approach to prevent selectively the protein expression of ERα or ERβ which allowed us to evaluate separately the contribution of ERα and ERβ on intracellular pathways in native endothelial and smooth muscle cells. Other investigators have used antisense gene therapy to decrease brain estrogen receptors.³² In their experiments, the intraventricular infusion of antisense decreased ER protein expression by 65% at 6 hours post-infusion. In our study, we observed that a treatment of PSMC or PAEC with selective antisense oligomers (10⁻⁶ mol/L) for 4 days decreased ERα and ERβ protein expression up to 97% (FIG. 1). ERα and ERβ can form homo and heterodimers in living cells.³³ By down regulating ERα or ERβ, we observed that 17βE can still induce its effects on vascular cells suggesting that the heterodimerization is not necessarily required.

### Biological Activities of 17βE Are Mediated Through ERβ in PSMCs

Usually activated by growth factors and cytokines, SMC proliferation and migration remain an important target to prevent in-stent restenosis. Many studies have indicated that estrogens prevent restenosis formation by inhibiting SMC proliferation and migration after balloon injury. We have previously demonstrated that local delivery of 17βE prevents restenosis upon an angioplasty.²⁷ In the current study, we observed that a treatment with 17βE (10⁻⁸ mol/L) inhibits PSMC migration and proliferation induced by PDGF-BB. In addition, the downregulation of ERβ protein expression reduced the inhibitory effects of 17βE on PSMC proliferation and migration. Our results support other studies suggesting that gene knockout of ERβ leads to hyperproliferative disease.³⁴ Recently, we have reported that a treatment of PSMC with 17βE reduces p42/44 and p38 MAPK phosphorylation induced by PDGF-BB.³⁰ To further evaluate the contribution of ERα and ERβ on PSMC, we demonstrated that a treatment with antisense oligomers targeting ERβ mRNA abrogated the inhibitory effects of 17βE on p42/44 and p38 MAPK phosphorylation mediated by PDGF-BB. These results support previous observations that ERβ may be responsible for an abnormal vascular contraction, ion channel dysfunction and hypertension in mice deficient in ERβ.²⁶ Lindner and co-workers have also demonstrated that ERβ mRNA expression is induced after vascular injury, supporting a direct contribution of this receptor in the vascular effects of estrogen.³⁵ In contrast to ERβ, the absence of ERα protein expression did not influence the inhibitory effects of 17βE on p42/44 and p38 MAPK phosphorylation in PSMC.

### ERα Activation by 17βE Induces MAPK Phosphorylation in PAECs

Various conditions such as hypercholesterolemia, hypertension, inflammation, and estrogen deficiency have been associated with endothelial dysfunction.¹⁸ The vessel wall impairment may contribute to the development of atherosclerosis and CVD. Several animal and in vitro studies have shown that estrogens improve endothelial function. We have demonstrated that local delivery of 17βE improves vascular healing and reendothelialization by promoting endothelial cell proliferation, migration and eNOS expression. However, the respective contribution of ERα and ERβ to these effects of 17βE has not been specifically evaluated. In the current study, we showed that the beneficial effects of 17βE on PAEC migration and proliferation are mediated through ERα stimulation. Our results are in agreement with the study of Brouchet et al,³⁶ who observed that ERα is required for estrogen-accelerated reendothelialization in an electric injury model. Estrogens can also interact with MAPK pathway³⁷ and we have previously demonstrated that 17βE induced significantly p42/44 and p38 MAPK activation on EC.³⁰ In the present paper, we showed that the inhibition of ERα protein expression reduces p42144 and p38 MAPK phosphorylation induced by 17βE. These results support previous work demonstrating a strong relationship between ERα activation by estrogens and MAPK activity in breast cancer cells.³⁸ Furthermore, our results confirm that the principal action of estrogen on endothelial cells are not mediated through ERβ. Ihionkhan and co-workers have postulated that estrogens upregulate ERα expression in endothelial cells supporting an important role of ERα for the biological effects of 17βE on the endothelium.³⁹

In conclusion, the properties of 17βE to promote p38 and p42/44 MAPK activation, migration and proliferation of PAEC are directly mediated through ERα stimulation. In contrast, 17βE inhibits these same events in PSMC which are mediated through ERβ activation. Our results suggest that in different vascular cell types but on the same mechanisms, effects of 17βE are not mediated through the same ER which may explain the distinct biological activity of estrogens. This study is providing new insights to our understanding on the specific contribution of estrogens on the vascular healing process.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

### REFERENCES

1. Cignarella A, Paoletti R, Puglisi L. Direct effects of estrogen on the vessel wall. Med Res Rev. 2001; 21:171-84.
2. Song J, Wan Y, Rolfe B E et al. Effect of estrogen on vascular smooth muscle cells is dependent upon cellular phenotype. Atherosclerosis. 1998; 140:97-104.
3. Lavigne M C, Ramwell P W, Clarke R. Inhibition of estrogen receptor function promotes porcine coronary artery smooth muscle cell proliferation. Steroids. 1999; 64:472-80.
4. Akishita M, Ouchi Y, Miyoshi H et al. Estrogen inhibits cuff-induced intimal thickening of rat femoral artery: effects on migration and proliferation of vascular smooth muscle cells. Atherosclerosis. 1997; 130:1-10.
5. Lantin-Hermoso R L, Rosenfeld C R, Yuhanna I S et al. Estrogen acutely stimulates nitric oxide synthase activity in fetal pulmonary artery endothelium. Am J. Physiol. 1997; 2731119-26.
6. Herrington D. Role of estrogens, selective estrogen receptor modulators and phytoestrogens in cardiovascular protection. Can J Cardiol. 2000; 16 Suppl E:5E-9E.
7. Cenci S, Weitzmann M N, Roggia C et al. Estrogen deficiency induces bone loss by enhancing T-cell production of TNF-alpha. J Clin Invest. 2000; 106:1229-37.
8. Silbiger S, Lei J, Ziyadeh F N et al. Estradiol reverses TGF-beta1-stimulated type IV collagen gene transcription in murine mesangial cells. Am J. Physiol. 1998; 274:F1113-8.
9. Mendelsohn M E, Karas R H. The protective effects of estrogen on the cardiovascular system. N Engl J. Med. 1999; 340:180111.
10. Hayashi K, Takahashi M, Kimura K et al. Changes in the balance of phosphoinositide 3-kinase/protein kinase B (Akt) and the mitogen-activated protein kinases (ERK/p38MAPK) determine a phenotype of visceral and vascular smooth muscle cells. J. Cell Biol. 1999; 145:727-40.
11. Simoncini T, Genazzani A R. Direct vascular effects of estrogens and selective estrogen receptor modulators. Curr Opin Obstet. Gynecol. 2000; 12:181-7.
12. Finking G. Krauss N, Romer S et al. 17-beta-estradiol, gender independently, reduces atheroma development but not neointimal proliferation after balloon injury in the rabbit aorta. Atherosclerosis. 2001; 154:39-49.
13. Giguere V, Tremblay A, Tremblay G B. Estrogen receptor beta: re-evaluation of estrogen and antiestrogen signaling. Steroids. 1998; 63:335-9.
14. Brzozowski A M, Pike A C, Dauter Z, Hubbard R E, Bonn T, Engstrom O, Ohman L, Greene G L, Gustafsson J A, Cariquist M. Molecular basis of agonism and antagonism in the oestrogen receptor. Nature. 1997; 389:753-8.
15. Mosca L, Manson J E, Sutherland S E, Langer R D, Manolio T, Barrett-Connor E. Cardiovascular disease in women: a statement for healthcare professionals from the American Heart Association. Writing Group. Circulation. 1997; 96:2468-2482.
16. Barrett-Connor E, Grady D. Hormone replacement therapy, heart disease, and other considerations. Annu Rev Public Health. 1998; 19:55-72.
17. Bush T L, Barrett-Connor E, Cowan L D, Criqui M H, Wallace R B, Suchindran C M, Tyroler H A, Rifkind B M. Cardiovascular mortality and noncontraceptive use of estrogen in women: results from the Lipid Research Clinics Program Follow-up Study. Circulation. 1987; 75:1102-1109.
18. Koh K K. Effects of estrogen on the vascular wall: vasomotor function and inflammation. Cardiovasc Res. 2002; 55:714-726.
19. Haynes M P, Li L, Russell K S, Bender J R. Rapid vascular cell responses to estrogen and membrane receptors. Vascul Pharmacol. 2002; 38:99-108.
20. Hulley S, Grady D, Bush T, Furberg C, Herrington D, Riggs B, Vittinghoff E. Randomized trial of estrogen plus progestin for secondary prevention of coronary heart disease in postmenopausal women. Heart and Estrogen/progestin Replacement Study (HERS) Research Group. Jama. 1998; 280:605-613.
21. Writing Group for the Women's Health Initiative Investigators. Risks and benefits of estrogen plus progestin in healthy postmenopausal women: principal results From the Women's Health Initiative randomized controlled trial. Jama. 2002; 288:321-333.
22. Luconi M, Forti G, Baldi E. Genomic and nongenomic effects of estrogens: molecular mechanisms of action and clinical implications for male reproduction. J Steroid Biochem Mol. Biol. 2002; 80:369-381.
23. Mendelsohn M E, Karas R H. The protective effects of estrogen on the cardiovascular system. N Engl J. Med. 1999; 340:1801-1811.
24. Tremblay G B, Tremblay A, Copeland N G, Gilbert D J, Jenkins N A, Labrie F, Giguere V. Cloning chromosomal localization, and functional analysis of the murine estrogen receptor beta. Mol. Endocrinol. 1997; 11:353-365.
25. Pare G, Krust A, Karas R H, Dupont S, Aronovitz M, Chambon P, Mendelsohn M E. Estrogen receptor-alpha mediates the protective effects of estrogen against vascular injury. Circ Res. 2002; 90:1087-1092.
26. Zhu Y, Bian Z, Lu P, Karas R H, Bao L, Cox D, Hodgin J, Shaul P W, Thoren P, Smithies O, Gustafsson J A, Mendelsohn M E. Abnormal vascular function and hypertension in mice deficient in estrogen receptor beta. Science. 2002; 295:505-508.
27. Chandrasekar B, Tanguay J F. Local delivery of 17-beta-estradiol decreases neointimal hyperplasia after coronary angioplasty in a porcine model. J Am Coll Cardiol. 2000; 36:1972-1978.
28. Chandrasekar B, Nattel S, Tanguay J F. Coronary artery endothelial protection after local delivery of 17beta-estradiol during balloon angioplasty in a porcine model: a potential new pharmacologic approach to improve endothelial function. J Am Coll Cardiol. 2001; 38:1570-1576.
29. Keshamouni V G, Mattingly R R, Reddy K B. Mechanism of 17-beta-estradiol-induced Erk1/2 activation in breast cancer cells. A role for HER2 AND PKC-delta. J Biol. Chem. 2002; 277:22558-22565.
30. Geraldes P, Sirois M G, Bernatchez P N, Tanguay J F. Estrogen regulation of endothelial and smooth muscle cell migration and proliferation: role of p38 and p42/44 mitogen-activated protein kinase. Arterioscler Thromb Vasc Biol. 2002; 22:1585-1590.
31. Arnal J F, Bayard F. Alteration in endothelial estrogen receptor expression: a potential key of vasculoprotection by estrogens? Circ Res. 2002; 91:759-760.
32. Inamdar S R, Eyster K M, Schlenker E H. Estrogen receptor-alpha antisense decreases brain estrogen receptor levels and affects ventilation in male and female rats. J Appl Physiol. 2001; 91:1886-1892.
33. Bai Y, Giguere V. Isoform-Selective Interactions between Estrogen Receptors and Steroid Receptor Coactivators Promoted by Estradiol and ErbB-2 Signaling in Living Cells. Mol. Endocrinol. 2003; 17:589-599.
34. Liu M M, Albanese C, Anderson C M, Hilty K, Webb P, Uht R M, Price R H, Jr., Pestell R G, Kushner P J. Opposing action of estrogen receptors alpha and beta on cyclin D1 gene expression. J Biol Chem. 2002; 277:24353-24360.
35. Lindner V, Kim S K, Karas R H, Kuiper G G, Gustafsson J A, Mendelsohn M E. Increased expression of estrogen receptor-beta mRNA in male blood vessels after vascular injury. Circ Res. 1998; 83:224-229.
36. Brouchet L, Krust A, Dupont S, Chambon P, Bayard F, Arnal J F. Estradiol accelerates reendothelialization in mouse carotid artery through estrogen receptor-alpha but not estrogen receptor-beta. Circulation. 2001; 103:423-428.
37. Levin E R. Cellular functions of plasma membrane estrogen receptors. Steroids. 2002; 67:471-475.
38. Zhang Z, Maier B, Santen R J, Song R X. Membrane association of estrogen receptor alpha mediates estrogen effect on MAPK activation. Biochem Biophys Res Commun. 2002; 294:926-933.
39. Ihionkhan C E, Chambliss K L, Gibson L L, Hahner L D, Mendelsohn M E, Shaul P W. Estrogen causes dynamic alterations in endothelial estrogen receptor expression. Circ Res. 2002; 91:814-820.

What is claimed is:

1. A method of modulating vascular healing after spontaneous, catheter or surgically induced injury in a mammal in need of such therapy, comprising the step of upregulating the expression of a gene encoding a mammalian ER selected from the group consisting of ERα and ERβ.

2. A method as described in claim 1, wherein the expression of ERα receptors is increased in endothelial cells.

3. A method as described in claim 1, wherein the expression of ERβ receptors is increased in smooth muscle cells.

4. A method as described in claim 1, further comprising the administration of 17βE or a related compound.

5. A method as described in claim 4, further comprising downregulation of ER that are different from those that are being upregulated.

6. A method of preventing atherosclerotic plaque vulnerability or destabilisation in a mammal in need of such therapy, comprising the step of upregulating the expression of a gene encoding a mammalian ER selected from the group consisting of ERα and ERβ.

7. A method as described in claim 6, wherein the expression of ERα receptors is increased in endothelial cells.

8. A method as described in claim 6, wherein the expression of ERβ receptors is increased in smooth muscle cells.

9. A method as described in claim 6, further comprising the administration of 17βE or a related compound.

10. A method as described in claim 9, further comprising downregulation of ER that are different from those that are being upregulated.

11. A method of treating atherosclerotic plaque vulnerability or destabilisation in a mammal in need of such therapy, comprising the step of upregulating the expression of a gene encoding a mammalian ER selected from the group consisting of ERα and ERβ.

12. A method as described in claim 11, wherein the expression of ERα receptors is increased in endothelial cells.

13. A method as described in claim 11, wherein the expression of ERβ receptors is increased in smooth muscle cells.

14. A method as described in claim 11, further comprising the administration of 17βE or a related compound.

15. A method as described in claim 14, further comprising downregulation of ER that are different from those that are being upregulated.

16. A method of reducing pathological angiogenesis in a mammal in need of such therapy, comprising the step of upregulating the expression of a gene encoding a mammalian ER selected from the group consisting of ERα and ERβ.

17. A method as described in claim 16, wherein the expression of ERα receptors is increased in endothelial cells.

18. A method as described in claim 16, wherein the expression of ERβ receptors is increased in smooth muscle cells.

19. A method as described in claim 16, further comprising the administration of 17βE or a related compound.

20. A method as described in claim 19, further comprising downregulation of ER that are different from those that are being upregulated.

21. A method of promoting saphenous vein graft healing in a mammal in need of such therapy, comprising the step of upregulating the expression of a gene encoding a mammalian ER selected from the group consisting of ERα and ERβ.

22. A method as described in claim 21, wherein the expression of ERα receptors is increased in endothelial cells.

23. A method as described in claim 21, wherein the expression of ERβ receptors is increased in smooth muscle cells.

24. A method as described in claim 21, further comprising the administration of 17βE or a related compound.

25. A method as described in claim 24, further comprising downregulation of ER that are different from those that are being upregulated.

26. A method of blocking pathological vascular injury or vulnerability in a mammal in need of such therapy, comprising the step of upregulating the expression of a gene encoding a mammalian ER selected from the group consisting of ERα and ERβ.

27. A method as described in claim 26, wherein the expression of ERα receptors is increased in endothelial cells.

28. A method as described in claim 26, wherein the expression of ERβ receptors is increased in smooth muscle cells.

29. A method as described in claim 26, further comprising the administration of 17βE or a related compound.

30. A method as described in claim 29, further comprising downregulation of ER that are different from those that are being upregulated.

31. A method of improving vascular healing in a mammal in need of such therapy, comprising the step of upregulating the expression of a gene encoding a mammalian ER selected from the group consisting of ERα and ERβ.

32. A method as described in claim 31, wherein the expression of ERα receptors is increased in endothelial cells.

33. A method as described in claim 31, wherein the expression of ERβ receptors is increased in smooth muscle cells.

34. A method as described in claim 31, further comprising the administration of 17βE or a related compound.

35. A method as described in claim 34, further comprising downregulation of ER that are different from those that are being upregulated.
